(19) United States
(12) Patent Application Publication
(10) Pub. No.: US 2004/0106871 A1
(43) Pub. Date: Jun. 3, 2004

(54) Determining the volume of a normal heart and its pathological and treated variants by using dimension sensors

(76) Inventors: Stephen Nicholas Hunyor, Gordon NSW (AU); Serguei Michael Plekhanov, NSW (AU); Yifei Huang, NSW (AU)

Correspondence Address: D Douglas Price, Steptoe & Johnson, 1330 Connecticut Avenue NW, Washington, DC, 20036, US

(21) Appl. No.: 10/468,801

(22) PCT Filed: Feb. 22, 2002
(86) PCT No.: PCT/AU02/00188

(30) Foreign Application Priority Data
Feb. 23, 2001 (AU) PR 3333

(51) Int. Cl. A61B 8/14; A61B 5/02

(52) U.S. Cl. 600/462; 600/508

(57) ABSTRACT

A method and system measure the instantaneous volume of blood contained within a chamber of a heart, irrespective of its shape, whereby stroke volume and cardiac output volume can be continuously monitored and feedback to a non-blood contacting cardiac assist device. In a preferred form the device uses the distances between the sensors which are implanted in a biomaterial that integrates with a heart surface to determine changes in heart volume. Sonomicrometry crystal measurements are disclosed as a preferred mode of obtaining distance readings. A computer readable medium carries instructions to convert data from dimension sensors into sensor positions within a predetermined coordinate system. Ventricular volume is based on the sensor positions.

### TECHNICAL FIELD

The present invention relates to an improved method of, system for, and computer readable medium of instructions for determining the volume or wall shape of a heart by utilising dimension sensors, and in particular, for determining the volume or wall shape of a heart which is diseased and may require the assistance of a cardiac assist device.

### BACKGROUND ART

Cardiac compression has been used to boost a failing heart for many years and in its most simple life-saving form involves the compression of the chest wall of a patient. In an emergency situation, a surgeon may take this one step further by manually compressing a heart that has failed, until recovery or an alternative treatment is instituted.

Of course, not all patients present in an acute state and typically a heart will be damaged over a period of time. This can also result in heart failure, a situation which occurs when the heart fails to maintain sufficient circulation of blood to provide adequate tissue oxygenation. Heart failure is widespread in the community affecting for example, 5 million Americans at any one time. Despite recent advances in cardiology, it remains on the increase.

Mechanical heart assist devices that can be used to boost an ailing heart have the potential to provide a quality of treatment that seriously challenges current treatment options, including heart transplantation. Whilst heart transplantation is effective in patients with severe heart failure, the shortage of donor hearts, the expense of the operation and post-operative care, and the risk of rejection are major drawbacks to this option ever fulfilling community expectations.

An assist device for a failing heart is described in International Patent Application No. PCT/AU00/00665. This specification discloses a heart actuator device for use in heart assist apparatus, the device including a patch-like main body, the main body including a heart compressing wall, which in use is adapted to be affixed to at least a region of the heart, and a distal wall, which in use is adapted to be distal that region of the heart, so as, in use to compress at least that region of the heart thereby assisting movement of the heart wall. The disclosures in International Patent Application No. PCT/AU00/00665 are incorporated herein by reference.

In certain situations, for example when utilising a cardiac assist device, distortion of ventricular chamber shape occurs, preventing the use of traditional geometry-based heart volume measurements.

Such volume and dimension measurements can be crucial for certain cardiac assist devices to be able to obtain an accurate correlation between the input parameters of a cardiac assist device and the volume or pressure of the blood which the failing heart with cardiac assist device pumps.

Presently, volume calculations using dimension sensors such as piezoelectric sonomicrometry crystals (crystals) are known. For example, some companies have designed software programs to exploit this technique.

Sonomicrometry gives data in the form of multiple distance measurements between any two crystals over time. If a sufficient number of crystals are used, and placed strategically, 3-D software can convert the matrix of distance measurements into the appropriate x, y, z coordinates for each crystal.

The surface area of a structure and the volume enclosed can be calculated with greater accuracy knowing the 3-D coordinates of the crystals. Previously, common practice was to strategically place at least two pairs of crystals (i.e. major and minor axis) on an object and assume that the structure the crystals enclose is a known geometrical shape (eg. in most cases ellipsoidal).

In the past, left ventricular (LV) heart volume measurements have been based on either 1, 2, or 3 axial measurements depending on the number and placement of crystals during the measurement. A number of different models are available to choose from.

Whole heart volumes can also be calculated using one, two, or three axis sonomicrometry measurements. The same models are available as the LV volume calculations.

Three different well-known volume model types have for several decades been selected for volume calculations. These three types are as follows:

1. Single Distance: This model type determines volume based on a single crystal pair measurement. The possible models to chose from based on single distance include:

$$Spherical Wall Volume = \frac{π {d}^{3}}{6}$$

Cubic Wall Volume=d³

$$Right Circular Cylinder Wall Volume = \frac{π {d}^{3}}{4} ( height = radius )$$

$$Paraboloid of Revolution Wall Volume = \frac{π {d}^{3}}{8} ( height = radius )$$

2. Double Distance: This model type determines volume based on two transmit/receive measurements. The possible models to chose from based on a double distance include:

Rectangular Wall Volume=d₁d₂²

$$Right Circular Cylinder Wall Volume = \frac{π}{4} {d}_{1} {d}_{2}^{2}$$

$$Ellipsoid Wall Thickness = \frac{π}{6} {d}_{1} {d}_{2}^{2}$$

$$Paraboloid of Revolution Wall Volume = \frac{π}{8} {d}_{1} {d}_{2}^{2}$$

3. Triple Distance: This model type determines volume based on three transmit/receive measurements. The possible models to chose from based on a triple distance include:

Rectangular Wall Volume=d₁d₂d₃

$$Frustum of Right Circular Cone Wall Volume = \frac{π}{12} {d}_{1} ( {d}_{2}^{2} + {d}_{2} {d}_{3} + {d}_{3}^{2} )$$

$$Ellipsoid Wall Thickness = \frac{π}{6} {d}_{1} {d}_{2} {d}_{3}$$

More recently, surface area and volume calculation software may use a ‘convex hull’ method and does not assume a particular geometrical shape or prefixed geometry. Therefore, it is a somewhat more accurate measure of absolute volume, surface area and wall shape, provided that a sufficient number of crystals are used.

This method necessarily requires that a convex geometry is assumed. Presently, this technique requires a minimum of 8 crystals for the left ventricle of a heart, and, 16 crystals for the right ventricle of a heart.

Although a large number of crystals can be utilised to obtain an accurate volume calculation using various techniques, such as those outlined above or numerical techniques, there is a need to provide increased accuracy in heart volume, surface area or wall shape calculations using only a relatively small number of crystals. By using a smaller number of crystals than is presently required, inconvenience to a patient can be reduced.

Furthermore, there is a need to provide accurate measurements of heart volume, surface area or wall shape calculations without the requirement of assuming a particular geometry, for example that a convex geometry exists.

Still furthermore, there is a need to provide tracking of real-time volume measurements and/or wall dynamics which can allow fine-tuning of compression efficiency and possible feedback control of the controller/driver for a cardiac assist device. By determining a more accurate model of the wall shape and/or volume measurement than is presently possible with a relatively small number of crystals these features can be realised or improved.

Also, the position of the crystals about the heart is important for accurate measurements. Positioning of crystals can be limited by, for example, surgeon access, whether the crystal implant procedure is open or closed chest, or a cardiac assist device itself. When utilising a relatively small number of crystals there is a requirement to place crystals in beneficial locations, for example preferably on the outside surface of the heart.

This identifies a need for determining heart volume, surface area or wall shape which overcomes or at least ameliorates the problems inherent in the prior art.

### DISCLOSURE OF INVENTION

The present invention seeks to provide a method of, system for and computer readable medium of instructions for quantitatively measuring the instantaneous volume of blood contained within a given chamber of the heart, whereby stroke volume and cardiac output volume can be continuously monitored and, if desired, used as a feedback signal for a cardiac assist device control system.

According to one aspect, the present invention aims to provide an improved means for determining the ventricular volume(s) of a mechanically assisted heart using sonomicrometry crystal measurements.

According to another aspect, the present invention also seeks to provide an improved means for measuring stroke volume and cardiac output volume with improved accuracy over that which has heretofore been possible using known prior art techniques.

In a specific embodiment, the present invention seeks to provide an improved means for measuring ventricular volume(s) using dimension sensors embedded in stand-alone passive patches or as active patches constituting part of a mechanically assisted heart. In one embodiment, the present invention also seeks to provide a means for using heart dimensions to control a mechanical cardiac assist device via a feedback loop.

In specific embodiments, the present invention seeks to provide a method, system or computer readable medium of instructions for heart volume calculation with at least some dimension sensors being crystals provided on heart patches which allows the number of crystals required to be reduced.

In a further embodiment of the present invention, the method, system or computer readable medium of instructions facilitates the continuous and instantaneous recording of left and right ventricular volumes, with a sampling frequency which is limited only by the analog-to-digital conversion rate.

In a specific embodiment of the present invention, there is provided a method of determining the ventricular volume(s) of a heart using dimension sensors attached to or placed about the heart, wherein at least one of the dimension sensors is embedded in at least one cardiac assist device which is attached to or placed about the surface of the heart, the ventricular volume(s) being calculated based on measured distances between selected dimension sensors and the shape of the at least one cardiac assist device.

In one form, the cardiac assist device is a heart patch which changes shape as a function of pneumatic or hydraulic pressure within the heart patch, the shape of the heart patch having a known correlation to the pressure within the heart patch. Preferably, the distances between selected dimension sensors, and the pressure inside the at least one heart patch, are simultaneously measured at a selected sampling rate. In a non-limiting embodiment, at least three dimension sensors are embedded in the at least one cardiac assist device. Also in a non-limiting embodiment, at least two dimension sensors are attached directly to the heart. Preferably, two heart patches are used.

In an alternate embodiment, one or more of the dimension sensors are provided in a passive heart patch which is attached to the surface of the heart. According to a particular aspect of one embodiment, some of the dimension sensors are implanted into a heart wall and/or the heart septum. According to a further particular aspect of one embodiment, eight, nine or ten dimension sensors are used for the volume calculation(s).

According to another particular aspect of one embodiment of the present invention, for each ventricle of the heart, geometrical shapes are fitted to the measured positions of selected dimension sensors, and the volume of each of the fitted geometrical shapes is used to calculate the ventricular volume of the heart. In a further possible form, at least one additional dimension sensor is used to obtain a thickness measurement of the left ventricle and/or right ventricle heart wall, the wall thickness measurement being factored into heart volume calculations. Preferably, the dimension sensors are piezoelectric devices or piezoelectric sonomicrometry crystals. In a particular embodiment, the measured signals from the piezoelectric sonomicrometry crystals are in the ultrasonic frequency range. Also preferably, the measured distances and volume calculations are performed in real-time.

In a further specific embodiment of the present invention, there is provided a method of assisting the function of a heart, the method including calculating heart stroke blood volume and cardiac output blood volume in real-time using crystals attached to or placed about the heart, wherein at least one of the crystals is embedded in at least one cardiac assist device which is attached to or placed about the surface of the heart, the heart stroke blood volume and cardiac output blood volume being calculated based on measured distances between selected crystals and the shape of the at least one cardiac assist device, which is known when distance measurements are obtained, and operation of the cardiac assist device being controlled as a function of the calculated heart stroke blood volume and cardiac output blood volume.

In a still further specific embodiment of the present invention, there is provided a method of determining the interior volume of a heart, the method including the steps of:

placing dimension sensors about interior and/or exterior surfaces of the heart, each dimension sensor able to receive and transmit signals;

determining a coordinate system to define the relative position of each dimension sensor;

determining a middle level plane, which intersects the left ventricle of the heart and the right ventricle of the heart, by using the position of at least three dimension sensors;

calculating the volume of a first region of the left ventricle, the first region of the left ventricle formed on a first side of the middle level plane, the calculation of the volume of the first region of the left ventricle based on measured distances between selected dimension sensors;

calculating the volume of a second region of the left ventricle, the second region of the left ventricle formed on a second side of the middle level plane, the calculation of the volume of the second region of the left ventricle based on measured distances between selected dimension sensors;

calculating the volume of a first region of the right ventricle, the first region of the right ventricle formed on a first side of the middle level plane, the calculation of the volume of the first region of the right ventricle based on measured distances between selected dimension sensors;

calculating the volume of a second region of the right ventricle, the second region of the right ventricle formed on a second side of the middle level plane, the calculation of the volume of the second region of the right ventricle based on measured distances between selected dimension sensors;

summing the volumes for each region of each ventricle thereby determining the volume of the heart as approximated by the location of the dimension sensors.

According to one possible aspect of the invention, a dimension sensor is provided on the right ventricle and/or a dimension sensor is provided on the left ventricle to enable wall thickness measurements to be factored into volume calculations.

According to a particular non-limiting embodiment, dimension sensor positions are located at or near the:

(i) apex; (ii) septum midlevel; (iii) centre left ventricle midlevel; (iv) anterior left ventricle midlevel; (v) centre left ventricle apical level; (vi) centre right ventricle midlevel; (vii) posterior right ventricle midlevel; and the (viii) centre right ventricle basal level, or the,

(i) apex; (ii) septum midlevel; (iii) centre left ventricle midlevel; (iv) posterior left ventricle midlevel; (v) centre left ventricle basal level; (vi) centre right ventricle midlevel; (vii) anterior right ventricle midlevel; and the (viii) centre right ventricle apical level.

In one further embodiment, the dimension sensors in at least positions (iii), (iv), (v), (vi), (vii) and (viii) are provided in the at least one cardiac assist device.

In a further specific embodiment of the present invention, there is provided a method of determining the wall shape of a heart using dimension sensors attached to or placed about the heart, wherein at least one of the dimension sensors is embedded in at least one cardiac assist device which is attached to or placed about the surface of the heart, the wall shape being calculated based on measured distances between selected dimension sensors and the shape of the at least one cardiac assist device, which is known when distance measurements were obtained.

In a still further specific embodiment of the present invention, there is provided a method of determining the wall shape of a heart, the method including the steps of:

placing dimension sensors about interior and/or exterior surfaces of the heart, at least some dimension sensors being attached to the heart and at least some dimension sensors provided within a cardiac assist device which is attached to the heart, each dimension sensor able to receive and transmit signals;

determining a coordinate system to define the relative position of each dimension sensor;

determining a middle level plane, which intersects the left ventricle of the heart and the right ventricle of the heart, by using the position of at least three dimension sensors;

fitting a geometrical surface to a first region of the left ventricle, the first region of the left ventricle formed on a first side of the middle level plane, the fitting calculation for the geometrical surface of the first region of the left ventricle based on measured distances between selected dimension sensors;

fitting a geometrical surface to a second region of the left ventricle, the second region of the left ventricle formed on a second side of the middle level plane, the fitting calculation for the geometrical surface of the second region of the left ventricle based on measured distances between selected dimension sensors;

fitting a geometrical surface to a first region of the right ventricle, the first region of the right ventricle formed on a first side of the middle level plane, the fitting calculation for the geometrical surface of the first region of the right ventricle based on measured distances between selected dimension sensors; and

fitting a geometrical surface to a second region of the right ventricle, the second region of the right ventricle formed on a second side of the middle level plane, the fitting calculation for the geometrical surface of the right ventricle based on measured distances between selected dimension sensors;

whereby the fitted geometrical surfaces approximate the wall shape of the heart.

In yet a further embodiment of the present invention, there is provided a system for assisting the function of a heart, the system including:

at least one dimension sensor for assisting in calculating heart volume in real-time, the at least one dimension sensor attached to a region of the heart;

at least one cardiac assist device, wherein further dimension sensors are embedded in the at least one cardiac assist device which is attached to or placed about the surface of the heart;

processing means in communication with each of the at least one dimension sensors attached to regions of the heart and the further dimension sensors embedded in the at least one cardiac assist device, the processing means receiving inter-sensor distance measurements;

a computer readable set of instructions associated with the processing means for calculating the heart volume based on measured distances between selected dimension sensors and the instantaneous shape of the at least one cardiac assist device; and

a cardiac assist device control unit which is in communication with the processor means, the cardiac assist device control unit controlling the functioning or operation of the at least one cardiac assist device based on the heart volume calculations performed by the computer readable set of instructions. The control unit can control hydraulic or pneumatic pressure to the heart patch.

In still yet a further embodiment of the present invention, there is provided a computer readable medium of instructions for determining the ventricular volume of a heart using dimension sensors attached to or placed about the heart, wherein at least one of the dimension sensors is embedded in at least one cardiac assist device which is attached to or placed about the surface of the heart, the instructions including procedures to:

receive data from the dimension sensors and data indicating the state of the at least one cardiac assist device;

convert the data into dimension sensor positions within a determined coordinate system; and

calculate the ventricular volume of the heart based on dimension sensor positions.

### BRIEF DESCRIPTION OF FIGURES

The present invention will become apparent from the following description, which is given by way of example only, of a preferred but non-limiting embodiment thereof, described in connection with the accompanying figures, wherein:

FIG. 1 (prior art) illustrates a heart patch;

FIG. 2 (prior art) illustrates heart patches in use on a heart;

FIG. 3 illustrates an horizontal cross-section of example crystal locations about a heart and heart patches;

FIG. 4 illustrates a vertical cross-section of example crystal locations which may be placed at any of the indicated various heights;

FIG. 5 illustrates a perspective view of possible crystal locations about a heart and heart patches;

FIG. 6 illustrates crystal locations in a preferred embodiment of the invention, the figure showing mid-level vertical and horizontal cross sections of the ventricular portions of a heart, crystals positions, and geometrical regions and lengths;

FIGS. 7a, 7b and 7c illustrate various geometrical parameters utilised for particular calculations in a particular embodiment of the present invention.

### BEST MODE FOR CARRYING OUT THE INVENTION

In a particular embodiment of the present invention there is provided a method of determining the left ventricular and right ventricular volumes of a mechanically assisted heart based on the instantaneous measurement of the distances between piezoelectric sonomicrometry crystals (crystals) embedded in a cardiac assist device and crystals implanted into or on the heart walls or heart septum.

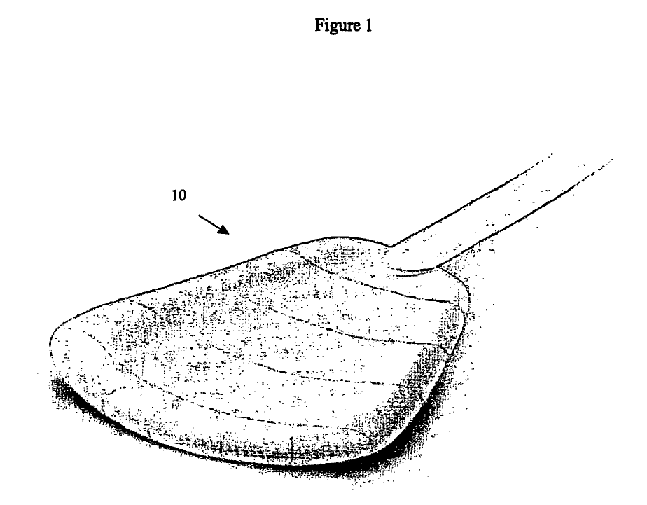

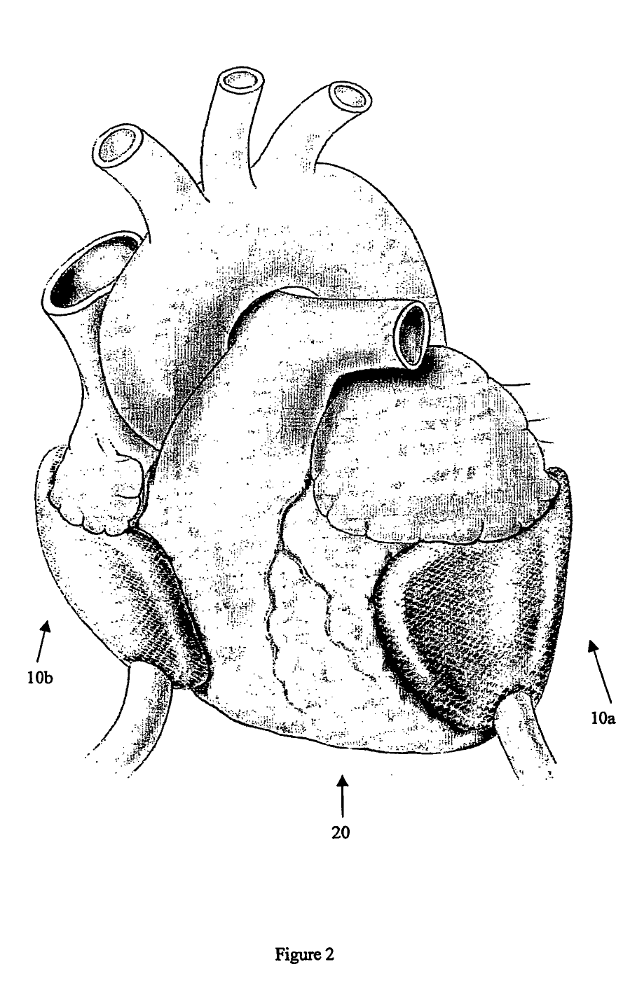

A cardiac assist device (heart patch 10) is presented in FIG. 1 as background information. Shown in FIG. 2 is a heart 20 with attached heart patches 10a and 10b, also as background information.

By placing crystals in selected locations about the heart (which may include within the heart), and having crystals embedded within the heart patches attached to the heart, an algorithm for calculating the volumes of the left and right ventricles of the heart can be used so that the heart volume, surface areas or heart wall geometries can be deduced.

In one particular embodiment, the present invention allows volume values during any number of heart cycles to be determined, whilst continuously calculating cardiac outputs and stroke volumes which can be provided as a difference between maximum and minimum values.

During contraction the heart undergoes complex 3-dimensional deformation. When a heart is impaired by disease its baseline shape and shape changes with contraction are disturbed. Additional, often marked shape changes are created by the use of mechanical heart assist devices such as the heart patch (a Direct Cardiac Compression ‘DCC’ device).

In accordance with a particular embodiment of the present invention, a method has been developed that allows reliable estimation of volume and volume changes in both the left and right ventricles in situations where the classical shape is disturbed. The method used, which is held to have a preferred application to the mechanically assisted heart, is based on measurements derived from piezoelectric crystals implanted in different regions of the myocardium and embedded into heart patches (a novel type of DCC device), although other devices may be utilised.

The crystals can be positioned on the heart at the apex and the interventricular septum midlevel. Moreover, three crystals can be attached to each heart patch (for LV midlevel: anterior, centre and basal; for RV midlevel: posterior, centre and apical, or vice versa).

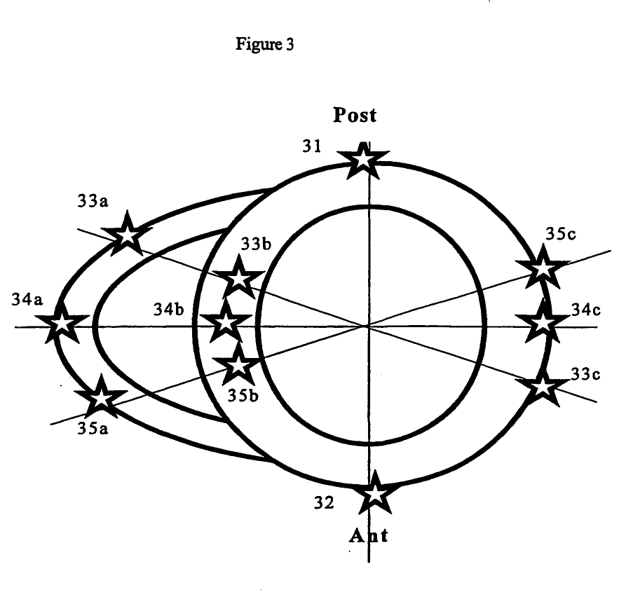

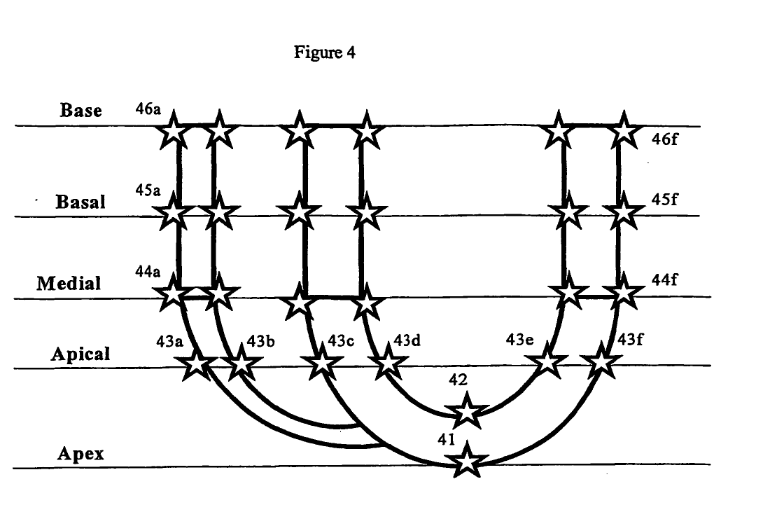

FIGS. 3 and 4 illustrate a variety of possible crystal locations shown from a horizontal-cross section and vertical cross-section through the heart respectively. FIG. 3 indicates a mid-level horizontal cross-sectional view showing possible crystal locations for different vertically-oriented planes. For example, crystals 31 and 32 could be used in conjunction with the sets of crystal 33a, 33b and 33c; or, 34a, 34b and 34c; or, 35a, 35b and 35c.

FIG. 4 indicates a mid-level vertical cross-sectional view showing possible crystal locations for different horizontally-oriented planes. For example, crystals 41 and 42 could be used in conjunction with the sets of crystal 43a, 43b, 43c, 43d, 43e and 43f; or, 44a, 44b, 44c, 44d, 44e and 44f; or, 45a, 45b, 45c, 45d, 45e and 45f; or, 46a, 46b, 46c, 46d, 46e and 46f.

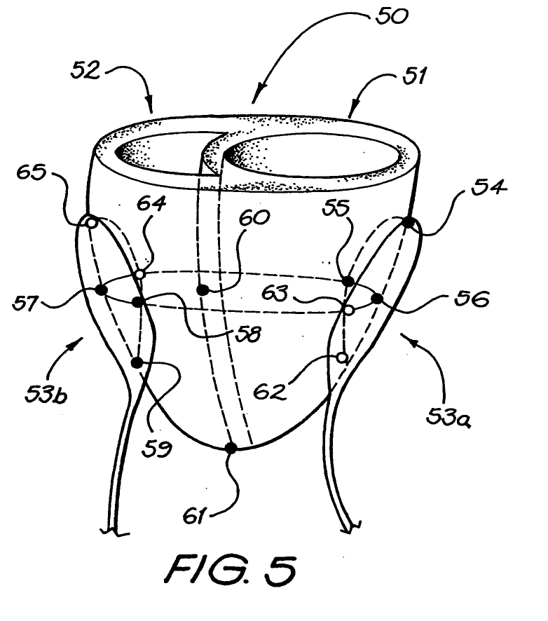

Shown in FIG. 5 is a perspective view of possible crystal locations about the heart and heart patches. Illustrated in FIG. 5 is a representation of a heart 50 having a left ventricle region 51 and a right ventricle region 52. A heart patch 53a is attached to left ventricle region 51, and a heart patch 53b is attached to right ventricle region 52. Crystals 54, 55 and 56 are embedded in heart patch 53a. Crystals 57, 58 and 59 are embedded in heart patch 53b. Crystal 60 is attached to the heart at the interventricular septum midlevel region, and crystal 61 is attached to the heart at the apex region. The positions on the heart patches 53a and 53b marked by the numerals 62, 63, 64 and 65 are extrapolated positions which can be calculated based on knowledge of the location of the real crystals in each heart patch and the instantaneous shape of each heart patch itself. Hence, knowledge of the positions of the eight crystals and the instantaneous shape of the heart patches themselves (which varies in use), in conjunction with a chosen coordinate system, can be used to calculate heart volumes.

It should be appreciated that the specific location and number of crystals can be widely varied. Also, each heart patch may contain more than three crystals, which may or may not be used in calculations. The heart patches used need not be the same overall shape, and other shaped heart patches could be utilised. The particular overall shape of heart patch used is not so important for the present invention, rather it is required that the shape of the heart patch at the time when crystal signals are obtained is known, so as to be able to perform the required calculations.

The distances between dimension sensors (sonodistances) are simultaneously recorded from all crystals, as well as the pressure inside each heart patch with an appropriate sampling rate. 3D coordinates are calculated for each crystal using three selected crystals from the heart patches as references for the x-y plane. Since the shape of heart patches is known, as well as the relationship between shape change and pressure change inside each heart patch, it is possible to interpolate or extrapolate coordinates of any point on cross-sections between the whole heart and left ventricle surfaces, including the septum, and parallel to the x-y plane and planes passing through a major axis.

Interpolation, or extrapolation, is possible because heart patches are attached to the heart surface by biointegrated material (making them integral “parts” of the heart surface) and the size and shape of heart patches is known. Interpolation is based on selection of the best-fitted curve for each sampling period from mathematical lines, for example a circle, ellipse, parabola, polynomial, logarithmic or straight line; or using B-spline interpolation.

Therefore, reconstruction of whole heart and left ventricle (LV) shell shape is provided. To derive right ventricle (RV) volume of the naturally beating heart and both RV and LV volumes of the mechanically assisted heart a pre-existing shell subtraction model can be applied.

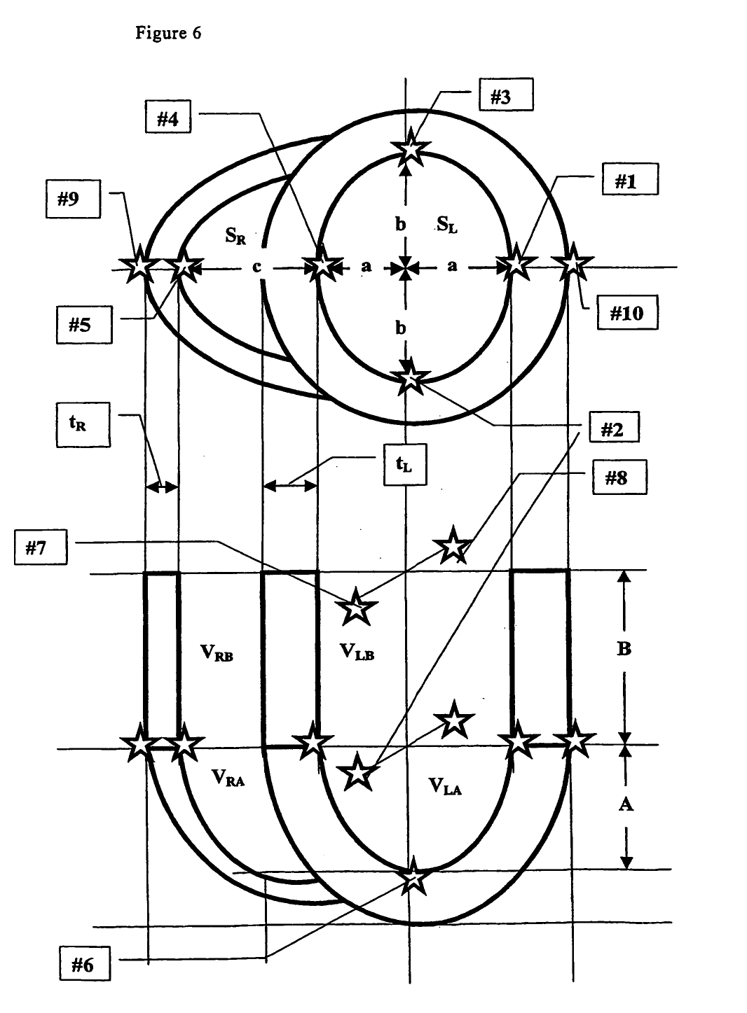

If crystals are placed into the epicardial (outer) surface then assumptions regarding free walls and septum thickness are made. The best method is to use endocardial (inner) placement. In this case it is required only to estimate the septum thickness (or place an additional crystal for a septum thickness measurement, for example crystal #9 or #10 with reference to FIG. 6).

Typically, it is not practical in humans to place the free wall crystals endocardially. However it may be possible to do so in experimental animals. The risk and trauma of endocardial placement (except for the septal crystals which may be delivered via the neck veins) is often too great in humans, however, this possibility remains.

### FURTHER EXAMPLE

Volume Model (Algorithm of Volume Calculation):

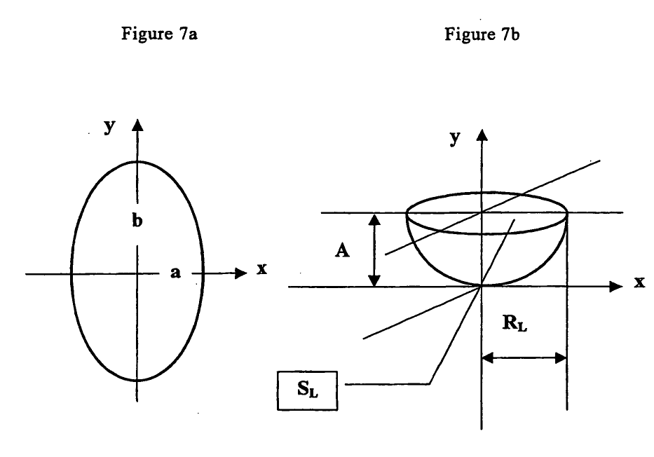

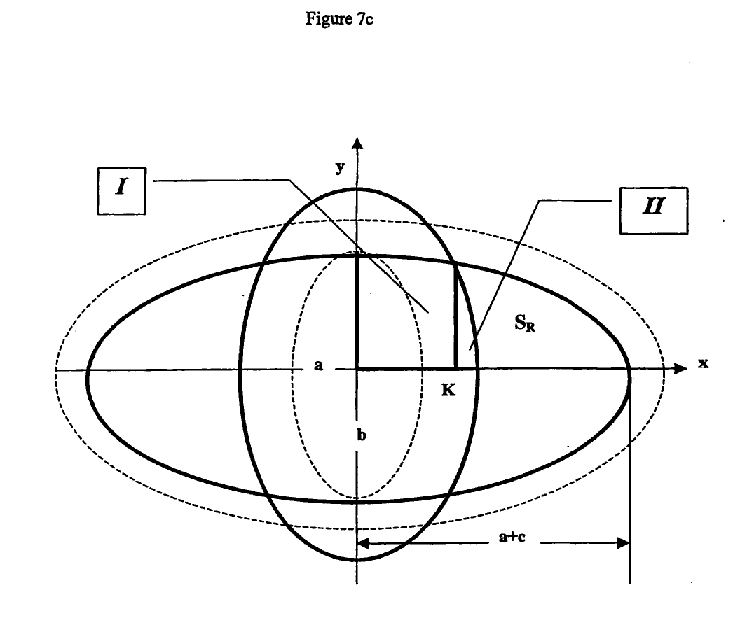

Calculations with ellipsoidal and paraboloidal shells are provided below with reference to FIGS. 6, 7a, 7b and 7c. This example is presented as one possible, but non-limiting, way of calculating heart volumes. It should be noted that various other geometrical shells can be assumed, but mathematical analysis is not herewith provided.

FIGS. 6, 7a, 7b and 7c illustrate real or virtual crystals and crystal positions as #1, #2, #3, #4, #5, #6, #7, #8, #9 and #10. Some crystals have real positions, the remaining positions are obtained by interpolation or extrapolation. The figures also show various geometrical parameters or variables used in the following calculation.

In determining the volume of the ventricles of a heart, crystals or virtual crystals #1 to #8 (and if desired #9 and/or #10) are placed about interior and/or exterior surfaces of the heart, each crystal able to receive and transmit signals. A coordinate system is determined to define the relative position of each crystal. A middle level plane is then determined, which intersects the left ventricle of the heart and the right ventricle of the heart, by using the position of at least three crystals.

The volume of a first region of the left ventricle V_{LA} is calculated, the first region of the left ventricle formed on a first side of the middle level plane, the calculation of the volume of the first region of the left ventricle obtained using the equation,

$${V}_{LA} = \frac{π abA}{2} ;$$

(eg. for paraboloidal approximation). The volume of a second region of the left ventricle V_{LB} is calculated, the second region of the left ventricle formed on a second side of the middle level plane, the calculation of the volume of the second region of the left ventricle obtained using the equation, V_{LB}=πabB; (eg. for cylindrical approximation). The volume of a first region of the right ventricle V_{RA} is calculated, the first region of the right ventricle formed on a first side of the middle level plane, the calculation of the volume of the first region of the right ventricle obtained using the equation,

$${V}_{RA} = \frac{π}{4} Ab ( 2 a + c ) - \frac{π}{2} ( a + {t}_{L} ) ( b + {t}_{L} ) ( A + {t}_{L} ) ;$$

(eg. for shell subtraction method). The volume of a second region of the right ventricle V_{RB} is calculated, the second region of the right ventricle formed on a second side of the middle level plane, the calculation of the volume of the second region of the right ventricle obtained using the equation,

$${V}_{RB} = \frac{π ( a + c ) b}{2} B - 2 B ( I + I I ) ,$$

(eg. shell subtraction method);

$$where , I = \frac{1}{2} b ( a + c ) [ {sin}^{- 1} ( \frac{K}{a + b} ) + \frac{K \sqrt{{( a + c )}^{2} - {K}^{2}}}{{( a + c )}^{2}} ]$$

$$and , \\ II = \frac{1}{2} ( b + {t}_{L} ) ( a + {t}_{L} ) [ {sin}^{- 1} ( \frac{a}{a + {t}_{L}} ) - {sin}^{- 1} ( \frac{K}{a + {t}_{L}} ) + \frac{a \sqrt{{( a + {t}_{L} )}^{2} - {a}^{2}}}{{( a + {t}_{L} )}^{2}} - \frac{K \sqrt{{( a + {t}_{L} )}^{2} - K}}{{( a + {t}_{L} )}^{2}} \\ and , \\ K = ( a + {t}_{L} ) ( a + c ) \sqrt{\frac{{t}_{L} ( 2 b + {t}_{L} )}{{( a + c )}^{2} {( b + {t}_{L} )}^{2} - {{b}^{2} ( a + {t}_{L} )}^{2}}}$$

The volume of the heart, V, is obtained as approximated by the location of the crystals using the equation,

V=V_{LB}+V_{LA}+V_{RB}+V_{RA};

wherein:

a=a(t)—first semi-axis of LV in middle level plane based on calculation a half distance between crystals #1 and #4;

b=b(t)—second semi-axis of LV in middle level plane based on calculation a half distance between crystals #2 and #3;

c=c(t)—axis of RV in middle level plane based on calculation a distance between crystals #4 and #5;

A=A(t)—long axis between middle plane and apex based on calculation distances between crystals #1, #2, #3, #4, #6;

B=B(t)—long axis between middle plane and base plane based on calculation distances between crystals #1, #2, #3, #4, #7, #8;

t_{L}—LV wall thickness or septum thickness is defined as constant at the diastole and systole with a linear or nonlinear growth factor, or can be calculated as a distance between additional crystal #10 and crystal #1.

In an alternate form of the present invention, the parameter t_{L} is not obtained by measurement but is defined to be a constant.

According to a further preferred embodiment of the present invention, a system is used for assisting the function of a heart and possible real-time monitoring of problems. After the crystals and heart patches are attached to desired regions of the heart, data signals from the crystals can be transmitted to a computer or other type of microprocessor-based monitoring device. The computer receives the signals from the crystals and a signal from a unit controlling the pressure in the heart patches. Software can be used to determine the shape of the heart patches based on known correlations between heart patch shape and pressure in the heart patch. Software can also be used to perform the volume or wall-shape calculations in accordance with the previously described method based on signals from the crystals indicating distance measurements.

Once the software has finished a cycle of calculations, for example of heart volume, the computer could communicate with the heart patch control unit to inform the control unit that the present functioning, mode of operation, or the like, of one or both of the heart patches should be altered. For example, volume calculations showing potential problems in heart stroke blood volume or cardiac output blood volume could be used to trigger the heart patch control unit to begin operation or increase/decrease pressure. By monitoring data signals in such a manner real-time control of heart patches can be realised.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

Although the preferred embodiment has been described in detail, it should be understood that various changes, substitutions, and alterations can be made herein by one of ordinary skill in the art without departing from the scope of the present invention as hereinbefore described and as hereinafter claimed.

1. A method of determining the ventricular volume(s) of a heart using dimension sensors attached to or placed about the heart, wherein at least one of the dimension sensors is embedded in at least one cardiac assist device which is attached to or placed about the surface of the heart, the ventricular volume(s) being calculated based on measured distances between selected dimension sensors and the shape of the at least one cardiac assist device.

2. The method as claimed in claim 1, wherein the cardiac assist device is a heart patch which changes shape as a function of pneumatic or hydraulic pressure within the heart patch, the shape of the heart patch having a known correlation to the pressure within the heart patch.

3. The method as claimed in claim 2, wherein the distances between selected dimension sensors, and the pressure inside the at least one heart patch, are simultaneously measured at a selected sampling rate.

4. The method as claimed in any one of the claims 1 to 3, wherein at least three dimension sensors are embedded in the at least one cardiac assist device.

5. The method as claimed in any one of the claims 1 to 4, wherein at least two dimension sensors are attached directly to the heart.

6. The method as claimed in any one of the claims 2 to 5, wherein two heart patches are used.

7. The method as claimed in any one of the claims 1 to 6, wherein one or more of the dimension sensors are provided in a passive heart patch which is attached to the surface of the heart.

8. The method as claimed in any one of the claims 1 to 7, wherein some of the dimension sensors are implanted into a heart wall and/or the heart septum.

9. The method as claimed in any one of the preceding claims, wherein eight, nine or ten dimension sensors are used for the volume calculation(s).

10. The method as claimed in any one of the preceding claims, wherein for each ventricle of the heart, geometrical shapes are fitted to the measured positions of selected dimension sensors, and the volume of each of the fitted geometrical shapes is used to calculate the ventricular volume(s) of the heart.

11. The method as claimed in any one of the preceding claims, wherein at least one additional dimension sensor is used to obtain a thickness measurement of the left ventricle and/or right ventricle heart wall, the wall thickness measurement being factored into heart volume calculations.

12. The method as claimed in any one of the preceding claims, wherein the dimension sensors are piezoelectric devices.

13. The method as claimed in any one of the preceding claims, wherein the dimension sensors are piezoelectric sonomicrometry crystals.

14. The method as claimed in claim 13, wherein the measured signals from the piezoelectric sonomicrometry crystals are in the ultrasonic frequency range.

15. The method as claimed in any one of the preceding claims, wherein the measured distances and volume calculations are performed in real-time.

16. A method of assisting the function of a heart, the method including calculating heart stroke blood volume and cardiac output blood volume in real-time using crystals attached to or placed about the heart, wherein at least one of the crystals is embedded in at least one cardiac assist device which is attached to or placed about the surface of the heart, the heart stroke blood volume and cardiac output blood volume being calculated based on measured distances between selected crystals and the shape of the at least one cardiac assist device, which is known when distance measurements are obtained, and operation of the cardiac assist device being controlled as a function of the calculated heart stroke blood volume and cardiac output blood volume.

17. The method as claimed in claim 16, wherein the cardiac assist device is a heart patch.

18. A method of determining the interior volume of a heart, the method including the steps of:
placing dimension sensors about interior and/or exterior surfaces of the heart, each dimension sensor able to receive and transmit signals;
determining a coordinate system to define the relative position of each dimension sensor;
determining a middle level plane, which intersects the left ventricle of the heart and the right ventricle of the heart, by using the position of at least three dimension sensors;
calculating the volume of a first region of the left ventricle, the first region of the left ventricle formed on a first side of the middle level plane, the calculation of the volume of the first region of the left ventricle based on measured distances between selected dimension sensors;
calculating the volume of a second region of the left ventricle, the second region of the left ventricle formed on a second side of the middle level plane, the calculation of the volume of the second region of the left ventricle based on measured distances between selected dimension sensors;
calculating the volume of a first region of the right ventricle, the first region of the right ventricle formed on a first side of the middle level plane, the calculation of the volume of the first region of the right ventricle based on measured distances between selected dimension sensors;
calculating the volume of a second region of the right ventricle, the second region of the right ventricle formed on a second side of the middle level plane, the calculation of the volume of the second region of the right ventricle based on measured distances between selected dimension sensors;
summing the volumes for each region of each ventricle thereby determining the volume of the heart as approximated by the location of the dimension sensors.

19. The method as claimed in claim 18, wherein the dimension sensors are attached to the heart and provided within a cardiac assist device which is attached to the heart.

20. The method as claimed in either claim 18 or 19, wherein a dimension sensor is provided on the right ventricle and/or a dimension sensor is provided on the left ventricle to enable wall thickness measurements to be factored into volume calculations.

21. The method as claimed in any one of the preceding claims, wherein dimension sensor positions are located at or near the:
(i) apex; (ii) septum midlevel; (iii) centre left ventricle midlevel; (iv) anterior left ventricle midlevel; (v) centre left ventricle apical level; (vi) centre right ventricle midlevel; (vii) posterior right ventricle midlevel; and the (viii) centre right ventricle basal level, or the,
(i) apex; (ii) septum midlevel; (iii) centre left ventricle midlevel; (iv) posterior left ventricle midlevel; (v) centre left ventricle basal level; (vi) centre right ventricle midlevel; (vii) anterior right ventricle midlevel; and the (viii) centre right ventricle apical level.

22. The method as claimed in claim 21, wherein the dimension sensors in at least positions (iii), (iv), (v), (vi), (vii) and (viii) are provided in the at least one cardiac assist device.

23. A method of determining the wall shape of a heart using dimension sensors attached to or placed about the heart, wherein at least one of the dimension sensors is embedded in at least one cardiac assist device which is attached to or placed about the surface of the heart, the wall shape being calculated based on measured distances between selected dimension sensors and the shape of the at least one cardiac assist device, which is known when distance measurements were obtained.

24. A method of determining the wall shape of a heart, the method including the steps of:
placing dimension sensors about interior and/or exterior surfaces of the heart, at least some dimension sensors being attached to the heart and at least some dimension sensors provided within a cardiac assist device which is attached to the heart, each dimension sensor able to receive and transmit signals;
determining a coordinate system to define the relative position of each dimension sensor;
determining a middle level plane, which intersects the left ventricle of the heart and the right ventricle of the heart, by using the position of at least three dimension sensors;
fitting a geometrical surface to a first region of the left ventricle, the first region of the left ventricle formed on a first side of the middle level plane, the fitting calculation for the geometrical surface of the first region of the left ventricle based on measured distances between selected dimension sensors;
fitting a geometrical surface to a second region of the left ventricle, the second region of the left ventricle formed on a second side of the middle level plane, the fitting calculation for the geometrical surface of the second region of the left ventricle based on measured distances between selected dimension sensors;
fitting a geometrical surface to a first region of the right ventricle, the first region of the right ventricle formed on a first side of the middle level plane, the fitting calculation for the geometrical surface of the first region of the right ventricle based on measured distances between selected dimension sensors; and
fitting a geometrical surface to a second region of the right ventricle, the second region of the right ventricle formed on a second side of the middle level plane, the fitting calculation for the geometrical surface of the right ventricle based on measured distances between selected dimension sensors;
whereby the fitted geometrical surfaces approximate the wall shape of the heart.

25. A system for assisting the function of a heart, the system including:
at least one dimension sensor for assisting in calculating heart volume in real-time, the at least one dimension sensor attached to a region of the heart;
at least one cardiac assist device, wherein further dimension sensors are embedded in the at least one cardiac assist device which is attached to or placed about the surface of the heart;
processing means in communication with each of the at least one dimension sensors attached to regions of the heart and the further dimension sensors embedded in the at least one cardiac assist device, the processing means receiving inter-sensor distance measurements;
a computer readable set of instructions associated with the processing means for calculating the heart volume based on measured distances between selected dimension sensors and the instantaneous shape of the at least one cardiac assist device; and
a cardiac assist device control unit which is in communication with the processor means, the cardiac assist device control unit controlling the functioning or operation of the at least one cardiac assist device based on the heart volume calculations performed by the computer readable set of instructions.

26. The system as claimed in claim 25, wherein the processing means is a computer or other device having a microprocessor.

27. The system as claimed in either claim 25 or 26, wherein the cardiac assist device is a heart patch and the control unit controls hydraulic or pneumatic pressure to the heart patch.

28. The system as claimed in any one of the claims 25 to 27, wherein the dimension sensors are sonomicrometry crystals.

29. A computer readable medium of instructions for determining the ventricular volume of a heart using dimension sensors attached to or placed about the heart, wherein at least one of the dimension sensors is embedded in at least one cardiac assist device which is attached to or placed about the surface of the heart, the instructions including procedures to:
receive data from the dimension sensors and data indicating the state of the at least one cardiac assist device;
convert the data into dimension sensor positions within a determined coordinate system; and
calculate the ventricular volume(s) of the heart based on dimension sensor positions.

30. The claim as claimed in claim 29, wherein the at least one cardiac assist device is at least one heart patch.

31. The claim as claimed in claim 30, wherein the data from the at least one heart patch is an indication of hydraulic or pneumatic pressure within the at least one heart patch, from which data the shape of the at least one heart patch can be determined.

32. The claim as claimed in either claim 30 or claim 31, wherein two heart patches are utilised, one attached to the left ventricle of the heart and the other to the right ventricle of the heart.

33. The claim as claimed in claim 32, wherein each heart patch contains at least three dimension sensors which are sonomicrometry crystals.
